(19) United States
(12) Patent Application Publication
(10) Pub. No.: US 2026/0183509 A1
(43) Pub. Date: Jul. 2, 2026

(54) METHOD OF ATTACHING A CONNECTOR ELEMENT TO A TUBULAR ELEMENT

(71) Applicant: Coloplast A/S, Humlebaek (DK)

(72) Inventors: Kim Bager, Lyngby (DK); Patrick Moritzen, Birkeroed (DK)

(21) Appl. No.: 19/130,548

(22) PCT Filed: Nov. 16, 2023
(86) PCT No.: PCT/DK2023/050280

§ 371 Date: May 16, 2025

(30) Foreign Application Priority Data
Nov. 16, 2022 (DK) PA 2022 70563

(51) Int. Cl. A61M 25/00

(52) U.S. Cl. CPC A61M 25/0014; A61M 2207/10

(57) ABSTRACT

The present disclosure relates to a method for attaching a connector element (13) to a tubular element (20) in a urinary catheter (10). The method comprising the steps of: providing a waveguide arrangement (30) extending between a first end (31) and a second end (32); arranging a distal portion (12) of the tubular element inside a receiving passage (23) of the connector element such that a portion of an outer surface (17) of the tubular element is positioned against a portion of an inner passage surface (24) of the receiving passage; and transmitting the optical radiation through the waveguide arrangement to deliver the optical radiation to the inner passage surface of the connector element such that a polymeric material at the inner passage surface is at least partly melted to thereby join at least a part of the portion of the inner passage surface to at least a part of the portion of the outer surface of the tubular element.

The present disclosure relates to a method of attaching a connector element to a tubular element in a urinary catheter, and a urinary catheter manufactured by the method.

### BRIEF DESCRIPTION OF THE DRAWING

The accompanying drawings are included to provide a further understanding of aspects and embodiments of the present disclosure and are incorporated into and a part of this specification. The drawings illustrate examples and together with the description serve to explain principles of aspects and examples. Other examples and many of the intended advantages of examples will be readily appreciated as they become better understood by reference to the following detailed description. The elements of the drawings are not necessarily to scale relative to each other. Like reference numerals designate corresponding similar parts.

FIG. 1 illustrates a cross-sectional view of a urinary catheter comprising a connector element and a tubular element,

FIG. 2 illustrates method steps according to an example of the present disclosure,

FIG. 3 illustrates a cross-sectional view of a connector element for a urinary catheter,

FIG. 4 illustrates a cross-sectional view of a part of a tubular element for a urinary catheter,

FIG. 5 illustrates a cross-sectional view of a waveguide arrangement for guiding optical radiation,

FIG. 6a-c illustrate attachment of a connector element to a tubular element by transmission of optical radiation through a waveguide arrangement,

FIG. 7a-h schematically illustrate various examples of waveguide arrangements and delivery of optical radiation, and

FIG. 8 schematically illustrates a manufacturing apparatus for attaching a connector element to a tubular element in a urinary catheter.

### DETAILED DESCRIPTION

In the following, various examples of the disclosure are described without reference to particular figures.

Examples of a first aspect of the present disclosure relate to a method for attaching a connector element to a tubular element in a urinary catheter, wherein the tubular element made from a first polymeric material and extends in an axial direction between a proximal portion and a distal portion, the tubular element comprising an inner surface and outer surface, the inner surface defining an interior lumen extending in the axial direction, wherein the distal portion comprises an opening providing access into the interior lumen,
wherein the connector element is made from a second polymeric material and comprises connector conduit between a connector opening and a receiving passage, the receiving passage having an inner passage surface,
wherein the method comprises the steps of:
   providing an elongated waveguide arrangement extending between a first end and a second end, the waveguide arrangement being configured to guide optical radiation between the first end and the second end to emit the optical radiation at the second end;
   arranging the distal portion of the tubular element inside the receiving passage of the connector element such that a portion of the outer surface of the tubular element is positioned against a portion of the inner passage surface of the connector element; and
   transmitting the optical radiation through the waveguide arrangement to deliver the optical radiation to the inner passage surface of the connector element such that the second polymeric material at the inner passage surface is at least partly melted to thereby join at least a part of the portion of the inner passage surface to at least a part of the portion of the outer surface of the tubular element.

The provision of delivery of optical radiation to the inner passage surface of the connector element via a waveguide arrangement to join the connector element to the tubular element may potentially improve manufacturing of urinary catheters.

In conventional manufacturing of urinary catheters, a distal portion of a tubular element is immersed in an adhesive and a connector element is then arranged onto that distal portion to thereby attach the connector element to the tubular element via the adhesive. An example of such an adhesive is cyclohexanone-based adhesive.

Such conventional methods may thereby require manufacturing workers to manually administer the adhesive, which can be a cumbersome task. Further, given that a urinary catheter is intended to be used as a medical device, the use of adhesives may impose additional rinsing, cleaning, or ventilation procedures onto the manufacturing process of the urinary catheter. In particular, the adhesive will typically enter an interior lumen of the tubular element when it is immersed in the adhesive and/or due to capillary action.

The method for attaching a connector element to a tubular element according to the present disclosure can avoid the use of adhesives. Thereby, the safety and health of manufacturing workers may be improved, and additional cleaning, rinsing, or ventilation steps can potentially be simplified or avoided. Furthermore, the resources required in manufacturing can be reduced, since the use of an adhesive can potentially be entirely avoided. Moreover, the bonding between the connector element and the tubular element is almost instant, in comparison with the bonding provided by an adhesive which may have to cure or harden for several hours.

A challenge in the development of new connection methods is the dimension of urinary catheters, which are relatively small.

Further, a tubular element is often at least partly transparent to, e.g., optical radiation in the visible spectrum, since it is desirable that a user of the catheter can visually inspect any contents of the tubular element. In contrast, the connector element is often at least partly opaque and may even have different colours for different versions of a urinary catheter, and accordingly, the connector element is ill-suited for transmission of optical radiation for welding. Given the typical condition that the portion of the outer surface of the tubular element which is in contact with the connecting element is typically concealed within the connector element, welding via optical radiation could conventionally be considered as unfeasible.

The inventors have, however, identified that optical radiation transmitted via a waveguide arrangement can accommodate the dimensions of a urinary catheter. By specifically targeting the inner passage surface of the connector element via the waveguide arrangement, the tubular element can be efficiently connected to the connector element despite the tubular element being potentially ill-suited for absorption and the connecting element being ill-suited from transmission of the optical radiation. The waveguide arrangement enables the optical radiation to be delivered specifically to the inner passage surface.

In examples of the present disclosure, the method comprises a step of inserting the second end of the waveguide arrangement into the interior lumen which is performed prior to the step of transmitting the optical radiation through the waveguide arrangement.

By inserting the second end of the waveguide arrangement into the interior lumen, the optical radiation can be accurately and efficiently delivered to the inner passage surface. In particular, the insertion of the waveguide arrangement permits the optical radiation to be provided at an angle of incidence which permits an efficient transmission of optical radiation through the inner surface of the tubular element. In contrast, optical radiation provided at a large angle of incidence may suffer from a relatively large fraction of the radiation being reflected.

Preferably, the second end of the waveguide arrangement is inserted into the tubular element such that the second end of the waveguide arrangement is positioned within the receiving passage of the connector element during the step of transmitting the optical radiation through the waveguide arrangement.

In examples of the present disclosure, the step of inserting the second end of the waveguide arrangement into the interior lumen is performed after the step of arranging the distal portion of the tubular element inside the receiving passage.

By inserting the waveguide arrangement into the interior lumen after arranging the tubular element inside the receiving passage of the connector element, the presence of a connector element may ease insertion of the waveguide arrangement. Typically, the connector conduit of the connector element may have an inner funnel surface terminating at the connector opening for the purpose of connecting the connector element with, e.g., a catheter bag. During manufacturing according to the present disclosure, this inner funnel surface may guide the waveguide arrangement into the distal portion of the tubular element in case the second end of the waveguide arrangement is misaligned with the axial direction of the tubular element prior to insertion.

In examples of the present disclosure, the second end of the waveguide arrangement is inserted into the interior lumen via the opening of the distal portion of tubular element.

The opening of the distal portion of the tubular element is located near the part of the portion of the outer surface of the tubular element which is to be welded. Hence, insertion of the second end of the waveguide arrangement through that opening can serve as a simple and efficient insertion procedure.

In examples of the present disclosure, the portion of the inner passage surface is joined to the portion of the outer surface in a continuous weld extending circumferentially around the outer surface of the tubular element.

A continuous weld advantageously provides a fluid-tight seal between the connector element and the tubular element. Preferably, the continuous weld extends fully around the outer surface of the tubular element.

In examples of the present disclosure, the first polymeric material and the second polymeric material comprise TPU.

TPU may also be referred to as thermoplastic polyurethane. This material can be melted to join the connector element to the tubular element, while simultaneously permitting the tubular element to be at least partly transparent while the connector element is opaque.

In examples of the present disclosure, the optical radiation is delivered to the inner passage in a transverse delivery orientation which is transverse to the axial direction of the interior lumen.

A transverse delivery orientation ensures that the optical radiation is delivered at an angle of incidence which permits efficient transmission of optical radiation through the inner surface of the tubular element.

Preferably, the optical radiation delivered to the inner passage in a transverse delivery orientation corresponds to at least 70% of the power being provided with an angle of incidence below 60 degrees, for example below 50 degrees, for example below 40 degrees, for example below 30 degrees, for example 20 degrees, such as below 10 degrees (relative to the normal of the inner surface). In this context, power is the amount of energy transferred per unit time, for example measured in units of watt, i.e., joule per second.

A transverse delivery orientation does not exclude optical radiation being delivered in more than one radial direction or being delivered circumferentially relative to the axial direction of the tubular element.

In examples of the present disclosure, the method comprises a step of performing a relative rotation around the axial direction of the tubular element during the step of transmitting the optical radiation through the waveguide arrangement.

Relative rotation around the axial direction of the tubular element may for example be implemented by rotation of the waveguide arrangement or the tubular element, such that the delivery of optical radiation is rotated around the axial direction relative to the tubular element and/or the connector element.

The provision of a relative rotation during the step of transmitting the optical radiation may ensure that a continuous and uniform weld is established around the entire circumference of the portion of the outer surface and the portion of the inner passage surface.

Performing a relative rotation is particularly relevant in examples in which the optical radiation is directed in one or more radial directions, i.e., where the optical radiation is not directed circumferentially within the receiving passage. However, relative rotation is not restricted to such examples. Even if optical radiation is redirected circumferentially within the tubular element, the rotation may improve the uniformity of the weld. This can for example be relevant in case the intensity of the optical radiation provided through the waveguide arrangement is not evenly distributed, in case of misalignment between the waveguide arrangement and an optical element, or in case of imperfect redirection by an optical element.

In examples of the present disclosure, the step of performing the relative rotation causes the transverse delivery orientation of the optical radiation to rotate relative to the outer surface of the tubular element and the inner passage surface of the connector element.

Accordingly, the outer surface of the tubular element and the inner passage surface is continuously joined.

Relative rotation causing the transverse delivery orientation to rotate may for example be implemented by rotation of the waveguide arrangement or rotation of the connector element and the tubular element.

In examples of the present disclosure, the step of performing relative rotation comprises either rotating the waveguide arrangement or the tubular element.

In case the tubular element is rotated, the connector element may typically also be rotated along with the tubular element.

In examples of the present disclosure, the method comprises:
providing an optical element configured to redirect the optical radiation;
inserting the optical element into the interior lumen such that the optical element is positioned within the receiving passage and the distal portion of the tubular element,
wherein the optical radiation is delivered to the inner passage surface by redirecting the optical radiation via the optical element after the optical radiation is transmitted through the waveguide arrangement.

The provision of an optical element positioned within the receiving passage allows the optical element to efficiently redirect optical radiation delivered thereto. In turn, the waveguide arrangement itself does not necessarily need to aim towards the inner passage surface, but merely has to transmit optical radiation to the receiving passage, from where the optical element can redirect the optical radiation accordingly.

Examples of viable optical elements are reflective elements such as mirrors, dispersive elements such as lenses and prisms, diffractive elements such as gratings, and diffusive elements such as engineered diffusers configured to diffuse and scatter optical radiation in a controlled manner. A metasurface can also constitute an optical element configured to redirect the optical radiation, for example by modulation of waves of the optical radiation through boundary conditions of the metasurface. Another example of an optical element is a waveguide tip assembly, such as a shaped optical fibre tip.

In examples of the present disclosure, the optical element is a waveguide tip assembly attached to the second end of the waveguide arrangement.

The provision of an optical element in the form of a waveguide tip assembly can ensure a simple and efficient redirection of optical radiation. In particular, an attachment between the waveguide arrangement and the optical element can ensure that the optical element is always correctly aligned with the waveguide assembly under use, and that the waveguide arrangement and the optical element can promptly be collectively inserted into the tubular element.

Examples of waveguide tip assemblies are shaped optical fibre tips, a mirror element attached to the second end of the waveguide arrangement, and a dispersive element attached to the second end of the waveguide arrangement.

In examples of the present disclosure, the optical element redirects the optical radiation from the axial direction of the interior lumen to the transverse delivery orientation.

Hence, the waveguide arrangement can transmit the optical radiation to the receiving passage of the connector element from where the optical element can redirect the optical radiation to the inner passage surface along a transverse delivery orientation.

In examples of the present disclosure, the optical element is conically shaped to circumferentially redirect the optical radiation radially.

A conically shaped optical element can circumferentially redirect the optical radiation radially, such that the inner passage surface of the connector element and the outer surface of the tubular element may potentially be joined along a weld extending circumferentially partially or fully around the outer surface of the tubular element. In contrast, a waveguide arrangement or an optical element which only delivers optical radiation in one or a few distinct radial directions may require, e.g., relative rotation to ensure a proper weld.

Examples of a conically shaped optical element is a conical mirror and an axicon lens.

In examples of the present disclosure, the optical element is configured to redirect the optical radiation directionally in at least one radial direction.

Preferably, the at least one radial direction is a distinct radial direction such that the optical radiation is not redirected circumferentially relative to the axial direction of the tubular element, but to one or more distinct radial directions. In comparison with optical radiation with is circumferentially redirected, this approach may reduce the power of the optical radiation required to melt the second polymeric material, since the optical radiation is distributed to a smaller area of the inner passage surface.

In examples of the present disclosure, the step of performing relative rotation comprises rotating the optical element.

Preferably, the optical element is rotated relative to the tubular element and the connector element.

In examples of the present disclosure, the first polymeric material is associated with a first attenuation coefficient of the optical radiation and the second polymeric material is associated with a second attenuation coefficient of the optical radiation, wherein the second attenuation coefficient is greater than the first attenuation coefficient.

In examples of the present disclosure, the second attenuation coefficient is greater than the first attenuation coefficient by a factor of at least 2, for example at factor of at least 5, such as a factor of at least 10.

As the first polymeric material has an attenuation lower than the second polymeric material, the optical radiation can efficiently be transmitted to the inner passage surface of the connector element. Further, since the second polymeric material has an attenuation greater than the first polymeric material, the second polymeric material may be capable of efficiently absorbing the optical radiation, and accordingly, it may be possible to avoid focusing optics configured to focus the optical radiation to a specific focal point near the interface between the connector element and the tubular element.

The attenuation coefficient may be understood as the (relative) attenuation of optical radiation per unit of distance of propagation in a given material.

In examples of the present disclosure, the optical radiation is only transmitted through the inner surface of the tubular element once after being emitted from the waveguide arrangement.

An alternative approach is to provide the optical radiation from outside the tubular element through the opening of the receiving passage of the connector element. By such an alternative approach, the optical radiation passes through the circumferential wall of the tubular element twice. For example, after transmission through the waveguide arrangement, the optical radiation will pass through the outer surface of the tubular element, the circumferential wall of the tubular element, the inner surface of the tubular element before entering the receiving passage of the connector element from where the optical radiation will once again pass through the inner surface and the circumferential wall of the tubular element before being delivered to the inner passage surface of the connector element. Each time the optical radiation passes through a surface, a fraction of the optical radiation is scattered.

In contrast, only transmitting the optical radiation through the inner surface once, i.e., one time, scattering of optical radiation prior to delivery to the inner passage surface can potentially be reduced. Such an approach may for example be implemented by inserting the second end of the waveguide arrangement into the interior lumen of the waveguide arrangement, from where optical radiation only has to pass through the inner surface of the tubular element once to arrive at the inner passage surface of the connector element.

In examples of the present disclosure, the second polymeric material of the connector element comprises an optically absorbent additive to provide the inner passage surface with an optical absorption band, wherein a centre wavelength of the optical radiation lies within the optical absorption band.

The provision of an optically absorbent additive added into or onto the second polymeric material of the connector element permits optical radiation to be efficiently absorbed primarily by the connector element.

In examples of the present disclosure, the optical absorption band has an upper boundary below 2000 nm, for example below 1600 nm, such as below 1200 nm.

The provision of an optically absorption band with an upper boundary permits the optical radiation to be adequately spaced to natural absorption peaks in the first polymeric material of the tubular element. Accordingly, the optical radiation can be transmitted through the tubular element and be efficiently absorbed by the connector element. This is particularly advantageous in the context of a urinary catheter, in which a smooth inner surface of the tubular element is desirable. If too much optical radiation is absorbed by the first polymeric material, the inner surface may be impaired.

An optical absorption band may for example be defined by a (second) attenuation coefficient of the second polymeric material. For example, the optical absorption band may be defined by the optical range in which a penetration depth of the optical radiation in the is below 1 mm. The penetration depth can be defined as the distance at which the intensity of optical radiation inside a material falls to 1/e, corresponding to approximately 37%, of its original value (due to absorption).

In examples of the present disclosure, a radius of the outer surface of the tubular element is greater than a radius of the inner passage surface of the connector element such that the connector element is press fitted onto the tubular element in the step of arranging the distal portion of the tubular element inside the receiving passage of the connector element.

In contrast to conventional manufacturing methods relying on the use of an adhesive, the present disclosure provides joining of a connector element and a tubular element through optical radiation. As a consequence thereof, previously employed dimensions of the connector element and the tubular element are not necessarily optimal.

When employing adhesive, a small gap between the two parts can be desirable to ensure that some quantity of adhesive can actually be present between the two parts. In contrast, a gap between the outer surface of the tubular element and the inner passage surface of the connector element is generally disadvantageous when welding and joining via optical radiation.

The provision of the radius of the outer surface of the tubular element being greater than the radius of the inner passage surface of the connector element ensures that any gap between the outer surface and the inner passage surface is minimized. In turn, this provision can also establish a press fit between the tubular element and the connector element, which can serve as a preliminary attachment between the connector element and the tubular element until these have been joined by welding through optical radiation. Accordingly, separate procedures for fixating the two parts to each other during transmission of optical radiation can be avoided.

The radius of the outer surface being greater than the radius of the inner passage surface can be complimented by the materials of the urinary catheter. Generally, polymeric materials suitable for urinary catheters, such as thermoplastic polyurethane, are relatively soft. Hence, it can be relatively simple to implement radii which minimize gaps between the relevant surfaces, and which provide an adequate press fit.

The radius of the outer surface of the tubular element can be measured from a central axis of the tubular element extending in the axial direction. The radius of the inner passage surface can be measured from a central axis of the receiving passage. When the tubular element and the connector element are attached with the distal portion of the tubular element arranged inside the receiving passage of the connector element, the central axis of the tubular element and the central axis of the receiving passage overlap. However, the radii should be measured when the tubular element and the connector element are not attached to each other.

Preferably, the radius of the outer surface of the tubular element is between 1% and 20% greater than the radius of the inner passage surface of the connector element, for example between 2% and 15%, such as between 3% and 10%.

Examples of a second aspect of the present disclosure relate to a urinary catheter manufactured by the method according to the first aspect of the present disclosure.

A urinary catheter manufactured by the method according to first aspect of the present disclosure may potentially avoid usage of adhesives such as cyclohexanone. In particular, the use of an adhesive can corrode, impair, or deteriorate the inner surface providing corrugations of that surface. Due to a lack of adhesive disposed on the inner surface of the tubular element, bacterial growth on the inner surface is easier to avoid, and the fluid flow may potentially be improved.

Examples of a third aspect of the present disclosure relate to a urinary catheter comprising:
a tubular element made from a first polymeric material and extending in an axial direction between a proximal portion and a distal portion, the tubular element comprising an inner surface and an outer surface, the inner surface defining an interior lumen extending in the axial direction, wherein the distal portion comprises an opening providing access into the interior lumen; and
a connector element made from a second polymeric material and comprising a connector conduit between a connector opening and a receiving passage, the receiving passage having an inner passage surface,
wherein the distal portion of the tubular element is arranged inside the receiving passage of the connector element such that a portion of the outer surface of the tubular element is positioned against a portion of the inner passage surface of the connector element, wherein at least a part of the portion of the inner passage surface of the connector element is joined to at least a part of the portion of the outer surface of the tubular element in a continuous weld extending circumferentially fully around the outer surface of the tubular element.

A urinary catheter in which the connector element is joined to the tubular element by a continuous weld can potentially provide several advantages compared to conventional urinary catheters in which the connector element is joined to the tubular element by an adhesive such as cyclohexanone. The content of the adhesive in the urinary catheter is naturally reduced. The use of an adhesive can potentially provide corrugations to the inner surface of the tubular element, and by reducing or omitting an adhesive, bacterial growth on the inner surface is easier to avoid, and the fluid flow may potentially be improved.

Typically, portion of the tubular element which is arranged inside the receiving passage of the connector has an axial length which is greater than the axial length of the continuous weld. As an example, the portion of the tubular element which is arranged inside the connector has an axial length which is 10.0 mm, whereas the axial length of the continuous weld is 2.0 mm.

Preferably, the continuous weld is displaced from an outer boundary of the receiving passage and displaced from an outer boundary of the distal portion of the tubular element. In other words, the continuous weld can be (axially) located centrally somewhere between, but not at, the outer boundary of the receiving passage and the outer boundary of the distal portion of the tubular element. Thereby, the continuous weld may potentially ensure that gaps between the outer surface of the tubular element and the inner passage surface of the connector element are not too large at the outer boundaries of the receiving passage and the distal portion. Such gaps may otherwise be prone to accumulation of bacteria or undesired debris.

In examples of the present disclosure, the inner surface of the tubular element is homogeneous along the axial direction.

This homogeneity may for example be quantified in terms of surface roughness and a surface roughness parameter. Of particular interest is that the surface roughness (parameter) of the inner surface along a portion of the tubular element arranged inside the receiving passage of the connector element is not substantially larger than the surface roughness (parameter) along a remaining portion of the tubular element along the axial direction.

Thereby, in preferable examples, a surface roughness parameter of the inner surface along a portion of the tubular element arranged inside the receiving passage is at most 50 percent greater than a surface roughness parameter of the inner surface along a remaining portion of the tubular element, for example at most 40 percent greater, for example 30 percent greater, for example 20 percent greater, for example 10 percent greater, such as 5 percent greater.

The surface roughness parameter can be measured according to the ISO 21920-2:2021 standard.

In examples of the present disclosure, the tubular element has not been exposed to an adhesive.

In examples of the present disclosure, the inner surface of the tubular element has not been exposed to an adhesive.

In examples of the present disclosure, the urinary catheter is manufactured by the methods according to the first aspect of the present disclosure.

Examples of a fourth aspect of the present disclosure relate to a manufacturing apparatus for attaching a connector element to a tubular element in a urinary catheter, wherein the tubular element is made from a first polymeric material and extends in an axial direction between a proximal portion and a distal portion, the tubular element comprising an inner surface and an outer surface, the inner surface defining an interior lumen extending in the axial direction, wherein the distal portion comprises an opening providing access into the interior lumen,
wherein the connector element is made from a second polymeric material and comprises a connector conduit between a connector opening and a receiving passage, the receiving passage having an inner passage surface, the manufacturing apparatus comprising:
   a catheter fixture configured to hold a catheter preassembly, the catheter preassembly comprising the connector element and the tubular element, wherein the distal portion of the tubular element is arranged inside the receiving passage of the connector element such that a portion of the outer surface of the tubular element is positioned against a portion of the inner passage surface of the connector element;
   an optical radiation source configured to emit optical radiation;
   an elongated waveguide arrangement extending between a first end and a second end, wherein the first end is optically coupled to the optical radiation source;
   a relative actuator configured to move the second end of the waveguide arrangement relative to the catheter fixture; and
   a controlling arrangement configured to control the relative actuator to insert the second end of the waveguide arrangement into the interior lumen when the catheter preassembly is held in the catheter fixture and configured to subsequently control the optical radiation source to transmit the optical radiation through the waveguide arrangement to deliver the optical radiation to the inner passage surface of the connector element.

The above-presented fourth aspect provides that a relative actuator is configured to move the second end of the waveguide arrangement relative to the catheter fixture. Within the scope of this provision, the relative actuator may for example move the second end of the waveguide arrangement, move the catheter fixture, or move both the second end of the waveguide arrangement and the catheter assembly. In each of these examples, the second end of the waveguide arrangement is moved relative to the catheter fixture. Regardless, such relative motion should preferably ensure that the second end of the waveguide arrangement can be inserted into the interior lumen of the tubular element when a catheter preassembly is held in the catheter fixture.

The above-presented fourth aspect further mentions a catheter preassembly. A catheter preassembly comprises the connector element and the tubular element in which the distal portion of the tubular element is arranged inside the receiving passage of the connector element such that a portion of the outer surface of the tubular element is positioned against a portion of the inner passage surface of the connector element. The catheter preassembly may thereby for example be a connector element press fitted onto the tubular element in which the connector element has not yet been joined to the tubular element by transmitting optical radiation to the inner passage surface.

A controlling arrangement can for example be implemented as a programmable logic controller such as an industrial microprocessor-based controller with programmable memory used to store program instructions and various functions. A controlling arrangement can be part of a larger industrial control system.

In examples of the present disclosure, the manufacturing apparatus is configured to attach the connector element to the tubular element by the method according to the first aspect of the present disclosure.

In the following, various concepts of the present disclosure are explained.

In the context of this disclosure, whenever referring to a proximal end or portion of the tubular element, the referral is to the end, which is closest to the user when the urinary catheter is in use. Whenever referring to a distal end or portion, the referral is to the end, which is furthest away from the user in use. The axial direction is the direction from the proximal to the distal end or vice-versa; i.e. for a urinary catheter, the axial direction corresponds to the longitudinal direction. The radial direction is the direction transverse to the axial direction.

In the context of this disclosure, a urinary catheter is a catheter suitable for being inserted into the urethra. The catheter typically comprises a tubular element with an outer diameter corresponding to the catheter size. The catheter size is typically between CH6 and CH18 for intermittent urinary catheters; CH stands for charrier and is a common indicator of catheter size. CH sizes indicates the outer diameter of the catheter, where the diameter is the number divided by three meaning that CH6 has an outer diameter of 2 mm and CH18 an outer diameter of 6 mm. The tubular element of the catheter is typically closed in a rounded off half-spherical tip in the proximal end; this is also known as a Nelaton tip. Other types of tips may be used, such as coude or Tiemann tip, which are tips that are at an angle with respect to the tubular element. Another example is so-called flex-tips, where the tip comprises a necked portion followed in the proximal direction by a rounded ball-shaped or olive-shaped portion. In the distal portion of the tubular element, the catheter may be provided with a connector element, which is adapted for connecting the catheter to a draining tube. The connector element typically has a funnel-shaped interior surface near the connector opening, such that a draining tube can be attached by a friction fit coupling. Generally, the connector element further has a receiving passage at which a distal portion of the tubular element is arranged when the urinary catheter has been manufactured.

The dimensions of a urinary catheter can vary depending on exact application, such as whether the urinary catheter is intended for male of female use or dependent on urologic conditions of the user. An exemplary length of a urinary catheter along the axial direction of a tubular element is 200 mm. Typical inner diameters of a tubular elements lie in the range from 1 mm to 4 mm, for example 1.2 mm, 1.7 mm, 2.3 mm, 2.7 mm, 3.2 mm, and 3.8 mm. In comparison, waveguide arrangements such as optical fibers can be provided with dimensions below 1 mm, thereby allowing insertion of such waveguide arrangements into the interior lumen of a tubular element.

Generally, if a waveguide arrangement is to be inserted into a tubular element, the waveguide arrangement may be inserted at either the proximal portion or the distal portion of the tubular arrangement. Depending on the manufacturing process, the tubular element may or may not be open at the proximal potion. On viable manufacturing procedure is to cut the tubular element from a tubular element stock, then the proximal portion of the tubular element is sealed and rounded, and subsequently, the proximal portion is provided with drainage openings or eyelets for draining urine from the bladder of a user. Given these conditions, a waveguide arrangement can in principle be inserted through the opening of the distal portion at any stage, at the opening of the proximal potion prior to sealing, or via the drainage openings or eyelets subsequently provided. However, typically, insertion through the opening of the distal portion is simplest to implement in practice.

An elongated waveguide arrangement may generally be referred to as a waveguide arrangement.

The urinary catheter of this disclosure may be of the intermittent type or the indwelling type. Common to both types of catheters is that they are inserted through the urethra until the tip reaches the bladder and urine starts to drain. The difference lies in the dwelling time in the bladder and retention means in case of indwelling catheters. An intermittent catheter only dwells in the bladder for as long as it takes to empty the bladder. These types of catheters preserve a normal bladder function in such a way that the bladder fills naturally and is emptied in a rhythm mimicking normal bladder emptying. An intermittent catheter is typically used 4 to 6 times per day. An indwelling catheter on the other hand is inserted into the bladder and retention means in the form of, e.g., an inflatable balloon or a malecot or other means are used to keep the catheter tip in the bladder for a period of days, weeks or even as long as a couple of months. During this dwelling period, the catheter continuously empties the bladder by allowing urine to drain through the catheter continuously. There will typically be a difference in the type of material used to make the tubular element between the two types of catheters. Typically, the intermittent catheter is somewhat rigid as compared to the indwelling catheter, because the intermittent catheter has to be inserted through the urethra a number of times per day without the insertion procedure causing too much effort, whereas the insertion of the indwelling catheter matters less, but rather the softness and pliability of the catheter during the dwelling period is of significance, so that it irritates the user as little as possible.

In examples, the urinary catheter may be provided with a hydrophilic coating.

The hydrophilic coating may be provided only on the insertable part of the catheter meaning that for example, the connector element is not coated. The hydrophilic surface coating is of the kind which, when hydrated or swelled using a swelling medium, reduces the friction on the surface area of the catheter which is intended to be inserted into the urinary channel of a user corresponding to the insertable part of the catheter.

An intermittent hydrophilic catheter differs from an indwelling catheter in that the hydrophilic surface coating of such a catheter is not suitable for indwelling use, because the surface coating tends to stick inside the mucosa of the urethra if left inside the body for a period exceeding 5-20 minutes, due to the hydrophilic coating transforming from being highly lubricious when fully wetted (95% weight water) to being adhesive when the hydration level of the coating is reduced (<75% weight water).

Generally, aspects of the present disclosure rely on joining an inner passage surface of a connector element to an outer surface of a tubular element using optical radiation. Since polymeric materials are used, the first polymeric material of the tubular element and the second polymeric material of the connector element can be efficiently joined or welded together while the two materials are arranged against each other.

Seen from the propagation path of the optical radiation, the second polymeric material is typically situated behind the first polymeric material. Typically, it is primarily the second polymeric material which absorbs the optical radiation. Accordingly, the optical radiation may preferably propagate through the first polymeric material with a relatively small part of the optical radiation being absorbed in this first polymeric material.

This can for example be achieved by having a first polymeric material which is at least partly transparent to the optical radiation while the second polymeric material is at least partly opaque to the optical radiation. Here, transparent and opaque should of course be understood relatively to the frequency and wavelength of the optical radiation, and not relatively to electromagnetic radiation which is visible to the human eye.

Proper absorption in the second polymeric material may for example be obtained by an adequately chosen optically absorbent additive embedded with the second polymeric material. Thereby, the second polymeric material can be tailored to absorb optical radiation while the first polymeric material does not.

Generally, a center wavelength of the optical radiation may preferably be in a range from 400 nm to 10,000 nm, more preferably in a range from 600 nm to 2,000 nm, even more preferably in a range from 800 nm to 1,200 nm.

Further, a power of the optical radiation may preferably be at least 5 W, for example at least 10 W, such as at least 30 W. Naturally, the required power of the optical radiation depends on the area to which it is distributed. Therefore, the required amount of optical radiation may alternatively be phrased in terms of a spatial peak intensity of the optical radiation at the inner passage surface. A spatial peak intensity of the optical radiation may preferably be at least 20 W/cm², for example at least 50 W/cm², such as at least 200 W/cm².

Such wavelengths, powers, and intensities can be obtained by employing a laser as an optical radiation source to provide the optical radiation. Suitable lasers are diode lasers, Nd:YAG lasers and CO₂ lasers. Typically, diode lasers are preferable, for example a diode laser of 808 nm or 940 nm wavelength.

Properly chosen power and wavelength can ensure that a part of the second polymeric material is melted. This can in turn melt a part of the first polymeric material. The melted parts of the first and second material join, and when delivery of optical radiation to the melted parts is terminated, the melted parts solidify to thereby establish a weld.

Generally, optical radiation is delivered to the inner passage surface of the connector element by using a waveguide arrangement to transmit the optical radiation. Such a waveguide arrangement may be any kind of structure which is suitable for guiding optical radiation from the first end and the second end of the waveguide arrangement. Since optical radiation is employed, examples of suitable waveguide arrangements include optical fiber waveguides, transparent dielectric waveguides, and a hollow tubular structure which is internally reflective to the optical radiation. A waveguide arrangement can also be, e.g., a bundle of fibers, for example arranged such that separate fibers can provide optical radiation to separate parts of the inner passage surface.

### DETAILED DESCRIPTION OF THE DRAWING

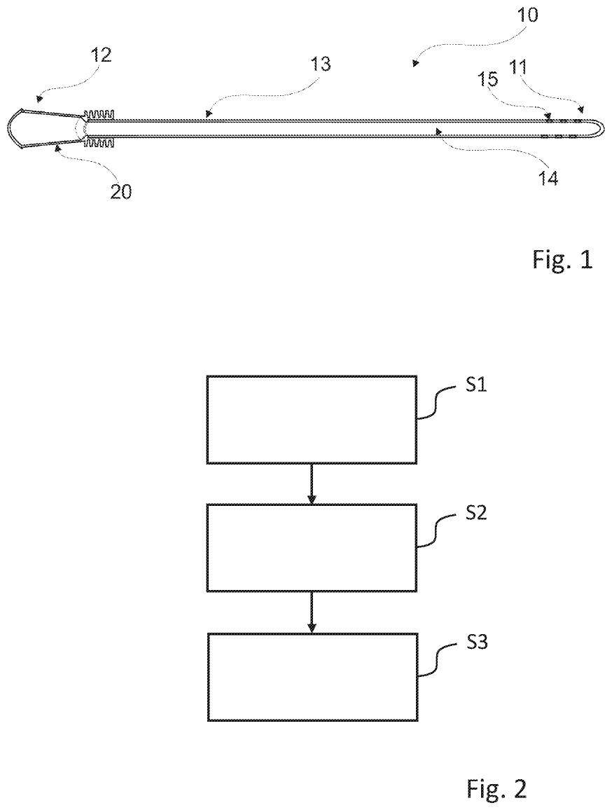

FIG. 1 illustrates a cross-sectional view of a urinary catheter 10 comprising a connector element 20 and a tubular element 13. The plane of the cross-sectional view lies along the longitudinal and axial direction of the tubular element 13 and of the urinary catheter 10.

The urinary catheter 10 has a proximal portion 11 adapted for being inserted into a urethra of a human and a distal portion 12 adapted for letting urine drain out of the catheter. The catheter has a tubular element 13 with an interior lumen 14 configured for draining urine from the proximal portion 11 to the distal portion 12. The proximal portion 11 has drainage openings 15 or eyelets such that urine from the bladder can enter through the drainage openings/eyelets into the interior lumen 14 and travel through this lumen 14 to the distal portion. A connector element 20 is attached at the distal portion 12 which can be used to connect, e.g., a draining tube.

FIG. 2 illustrates method steps S1-S3 according to an example of the disclosure. The example relates to attaching a connector element to a tubular element.

In this example, the tubular element is made from a first polymeric material and extends in an axial direction between a proximal portion and a distal portion. The tubular element comprises an inner surface and an outer surface. The inner surface defines an interior lumen extending in the axial direction. The distal portion comprises an opening providing access into the interior lumen.

Further, the connector element is made from a second polymeric material and comprises a connector conduit between a connector opening and a receiving passage. The receiving passage has an inner passage surface.

In a first step S1 of the method, an elongated waveguide arrangement is provided. The waveguide arrangement extends between a first end and a second end and is configured to guide optical radiation between the first end and the second end to emit this optical radiation at the second end.

As an example, the waveguide arrangement can be provided together with an optical radiation source configured to emit the optical radiation. Then, the first end of the waveguide arrangement is typically optically coupled to the optical radiation source, such that optical radiation emitted from the optical radiation source is optically coupled into the first end, transmitted through the waveguide arrangement, and emitted at the second end.

The waveguide arrangement (and, optionally, the optical radiation source) can be provided as part of a partially or fully automized manufacturing apparatus.

In a next step S2 of the method, the distal portion of the tubular element is arranged inside the receiving passage of the connector element such that a portion of the outer surface of the tubular element is positioned against a portion of the inner passage of the surface of the connector element.

This step can be performed manually or automatically. In case of manual performance, a human operator can simply insert the distal portion into the receiving passage. In case of automated performance, a linear actuator or other machinery can be employed to insert the distal portion into the receiving passage.

A tubular element and a connector element with the distal portion of the tubular element arranged inside the receiving passage of the connector element without a weld in between constitutes catheter preassembly.

In a next step S3 of the method, optical radiation is transmitted though the waveguide arrangement to thereby deliver the optical radiation to the inner passage surface of the connector. This delivery of optical radiation causes the second polymeric material at the inner passage surface to at least partly melt thereby joining at least a part of the portion of the inner passage surface to at least a part of the portion of the outer surface of the tubular element.

Accordingly, the connector element and the tubular element are attached by welding.

Optionally, methods according to examples of the present disclosure may further comprise a step of inserting the second end of the waveguide arrangement into the interior lumen of the tubular element. This step is then performed prior to the step of transmitting optical radiation through the waveguide arrangement.

This step of inserting the second of the waveguide arrangement into the interior lumen may be performed manually or automatically. In case of automated performance, a relative actuator can be employed which is configured to move the second end of the waveguide arrangement and/or the tubular element.

The step of transmitting optical radiation may be initiated manually or automatically. An automated initiating may for example be performed in response to inserting the second end of the waveguide arrangement into the interior lumen.

Further, methods according to examples of the present disclosure may optionally comprise a step of performing a relative rotation around the axial direction of the tubular element during the step of transmitting the optical radiation through the waveguide arrangement. Such a step may be performed manually or automatically. If performed automatically, it may for example be implemented by a rotary actuator, such as a stepper motor.

Generally, any automated performance of method steps may, for example, be controlled by a controlling arrangement.

Note that, generally, fully manufacturing a urinary catheter comprises additional steps such as moulding, coating, cutting, etc.

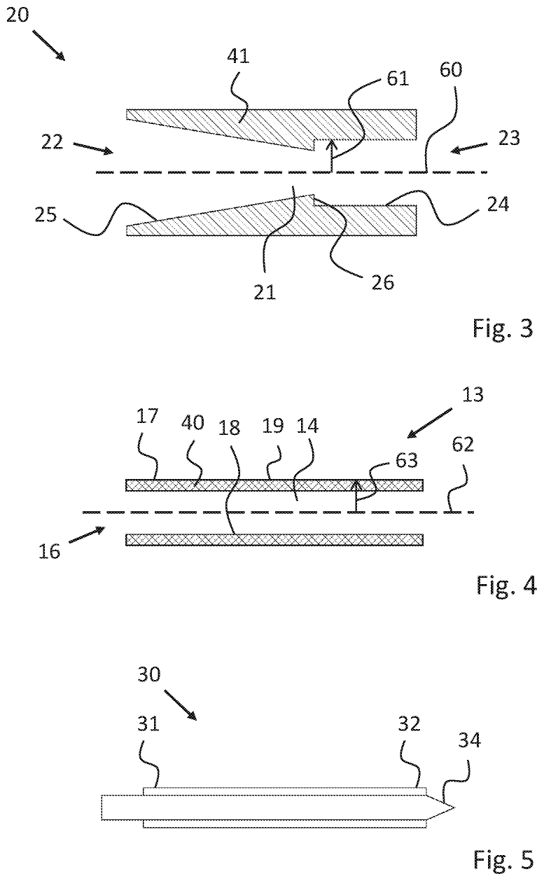

FIG. 3 illustrates a cross-sectional view of a connector element 20 for a urinary catheter.

The connector element 20 is formed by a second polymeric material 41. In this specific example, the second polymeric material 41 is constituted by a thermoplastic polyurethane.

The connector element 20 comprises a connector conduit 21 which fluidly connects a connector opening 22 and a receiving passage 23. The receiving passage 23 is configured to receive a tubular element, and the connector opening 22 is configured to facilitate draining of urine from the manufactured urinary catheter. The receiving passage 23 has an inner passage surface 24 which terminates at an inner abutment 26 which thereby defines an obstructing stop for a tubular element being inserted into the connector element 20. The connector conduit 21 has an inner funnel surface 25 for connecting the opening 22 with, e.g., a draining tube.

The receiving passage 23 and the inner passage surface 24 define a central axis 60 of the receiving passage 23. In this particular example, the inner passage has a cylindrical shape, in which the central axis 60 corresponds to the axis of this cylinder. From this central axis 60, a radius 61 of the inner passage surface 24 can be measured.

FIG. 4 illustrates a cross-sectional view of a part of a tubular element 13 for a urinary catheter. In this particular illustration, only the part of the tubular element near the distal portion 17 is illustrated.

The tubular element 13 is formed by a first polymeric material 40. In this specific example, the first polymeric material 40 is constituted by a thermoplastic polyurethane.

The tubular element 13 extends in an axial direction between a proximal portion (not shown) and a distal portion 17. In the illustration, the axial direction overlaps with a central axis 62 of the tubular element 13.

The tubular element 13 further has an inner surface 18 and an outer surface 17, wherein the inner surfaces 18 defines an interior lumen 14 configured to facilitate drainage of urine through the tubular element 13 when the urinary catheter is in use.

The outer surface 19 defines the central axis of the tubular element 62. In this particular example, the tubular element 13 is shaped as a hollow cylinder, in which the central axis 62 corresponds to the axis of this hollow cylinder. From this central axis 62, a radius 63 of the outer surface 19 of the tubular element 13 can be measured.

FIG. 5 illustrates a cross-sectional view of a waveguide arrangement 30 for guiding optical radiation. The waveguide arrangement 30 extends between a first end 31 and a second 32, and the arrangement 30 is configured to guide optical radiation from the first end 31 to the second end 32.

In this particular example, the waveguide arrangement 30 comprises an optical element 34 configured to redirect optical radiation. The waveguide arrangement 30 is integrally attached to this optical element 34, which is here implemented as a waveguide tip assembly in the form of a conical tip configured to circumferentially redirect optical radiation radially, when the optical radiation is transmitted through the waveguide arrangement 30 from the first end 31 to the second end 32. Hence, a laser beam of optical radiation transmitted through the waveguide arrangement 30 may be outputted circumferentially at the optical element 34 in a ring pattern.

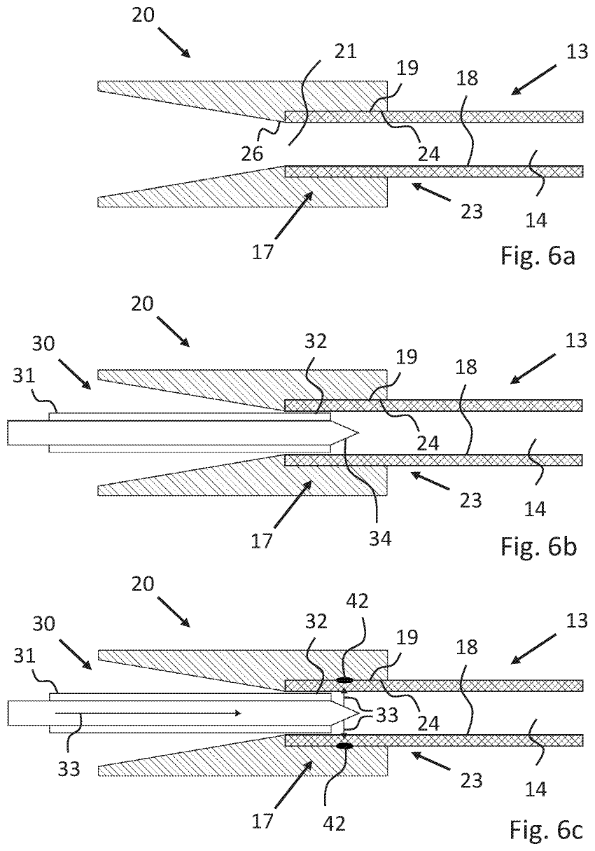

FIG. 6a-c illustrate attachment of a connector element 20 to a tubular element 13 by transmission of optical radiation 33 through a waveguide arrangement 30. The connector element 20 is substantially similar to the connector element illustrated in FIG. 3, the tubular element 13 is substantially similar to the tubular element 13 illustrated in FIG. 4, and the waveguide arrangement 30 is substantially similar to the waveguide arrangement illustrated in FIG. 5.

In FIG. 6a, the tubular element 13 is preliminarily attached to the connector element 20 to form a catheter preassembly. The connector conduit 21 of the connector element 20 has an inner abutment 26 protruding radially inward and defining an obstructing stop for the tubular element 13. Accordingly, the distal portion 17 of the tubular element 13 is inserted into the receiving passage 23 of the connector element 20, such that the tubular element 13 terminates at the inner abutment 26.

In this particular example, the connector element 20 is preliminarily attached to the tubular element 13 by press fit. This fit is enabled by the radius of the outer surface of the tubular element 13 being slightly larger than the radius of the inner passage surface.

In FIG. 6b, the waveguide arrangement 30 is inserted into the interior lumen 14 of the tubular element 13. The waveguide arrangement 30 is inserted such that the second end 32 of the waveguide arrangement 30 is located within the distal portion 17 of the tubular element 13 and located within the receiving passage 23 of the connector element 20.

Moreover, in this example in which the waveguide arrangement 30 has an optical element 34, the optical element is also located within the distal portion 17 of the tubular element 13 and located within the receiving passage 23 of the connector element 20.

In FIG. 6c, optical radiation 33 is transmitted through the waveguide arrangement 30 to deliver the optical radiation 33 to the inner passage surface 24 of the connector element 20. The optical radiation 33 may for example be provided from an optical radiation source such as a laser source.

The optical radiation 33 transmitted through the waveguide arrangement 30 propagates along a longitudinal axial direction of the waveguide arrangement 30, which in this illustration overlaps with the central axis of the receiving passage and the central axis of the tubular element. At the second end 32 of the waveguide arrangement 30, the optical radiation 33 is outputted to be delivered to the inner passage surface 24 of the connector element 20. In this particular example, the optical radiation 33 is redirected at the second end 32 by an optical element 34 from the axial direction to a transverse delivery orientation, transverse to the axial direction of the interior lumen.

In this particular illustration, the redirection by the optical element is illustrated such that the propagation direction of the optical radiation before and after the redirection forms a right angle. However, note that redirection of optical radiation to a transverse delivery orientation can be performed at any angle as long as the resulting transverse delivery orientation is at least partially transverse to the axial direction of the interior lumen.

The optical radiation 33 is transmitted through the tubular element 13 and delivered to the inner passage surface 24 of the connector element. The second polymeric material of the connector element absorbs the optical radiation, such that the second polymeric material at a part of the portion of the inner passage surface is melted to form melted polymeric material 42. Typically, heat is also transferred to the first polymeric material at the outer surface 19 of the tubular element 13, for example heat from the melted polymeric material 42 formed from the second polymeric material of the connector element 20. Thereby, some of the first polymeric material can also be mixed into the melted polymeric material 42, such that the melted polymeric material 42 comprises both the first polymeric material and the second polymeric material. Regardless, the melted polymeric material 42 is typically primarily formed by the second polymeric material.

When a sufficient amount of polymeric material is melted, the transmission of optical radiation 33 is terminated, so that the melted polymeric material 42 can solidify to form a weld between the connector element and the tubular element.

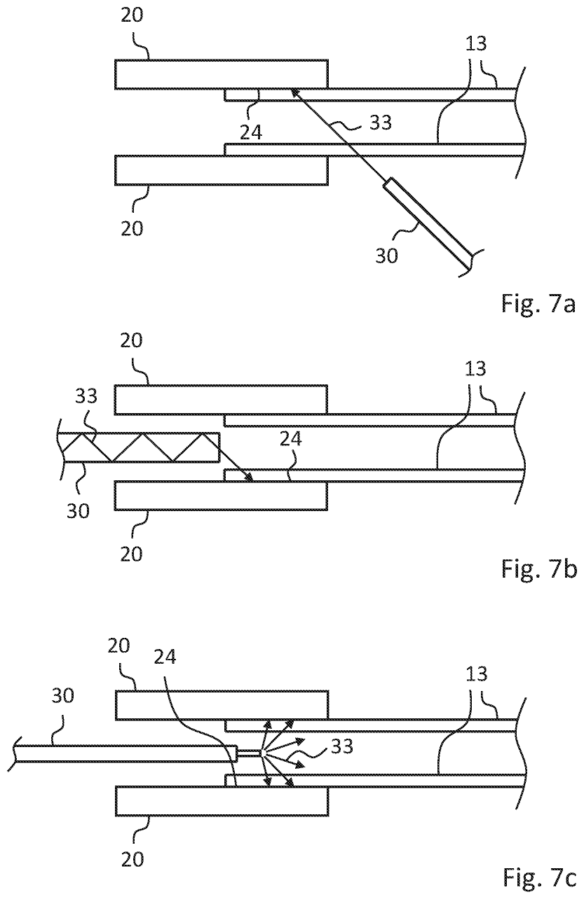

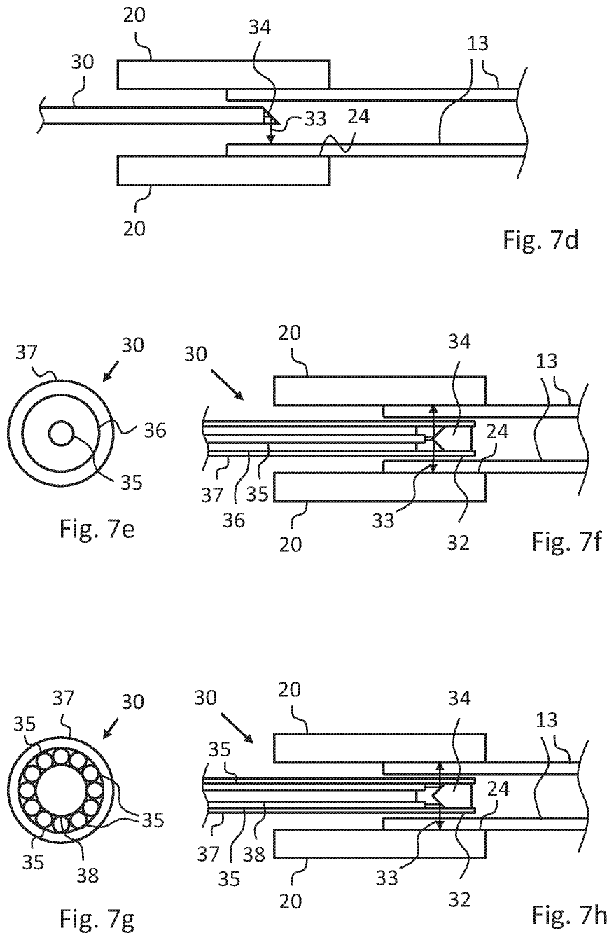

FIG. 7a-h schematically illustrate various examples of waveguide arrangements and delivery of optical radiation.

FIG. 7a illustrates a waveguide arrangement 30 arranged outside the connector element 20 and the tubular element 13 delivering optical radiation 33 to the inner passage surface 24 of the connector element 20. The optical radiation is delivered through the opening of the receiving passage of the connector element. The waveguide arrangement can for example be an optical fibre, for example with a collimating or focusing outcoupling lens for providing sufficiently intense optical radiation at the inner passage surface 24.

FIG. 7b illustrates a waveguide arrangement 30 arranged outside to tubular element 13 to deliver optical radiation 33 at the inner passage surface of the connector element 20. In this example, the waveguide arrangement 30 relies on internal reflection to deliver the optical radiation 33 at a transverse delivery orientation which is transverse to the axial direction of the interior lumen. In practice, such a waveguide arrangement 30 may be implemented by a rod having an internal reflective surface, or by a dielectric material capable of providing total internal reflection of optical radiation propagating with an angle of incidence relative to a circumferential surface of the dielectric material with is greater than a critical angle. As an example, the critical angle for acrylic glass in air is approximately 42 degrees for optical radiation having a wavelength of 589 nm.

FIG. 7c illustrates a waveguide arrangement 30 providing diverging optical radiation 33. The optical radiation 33 is provided within the distal portion of the tubular element 13. The waveguide arrangement may for example be an optical fibre provided with a waveguide tip assembly in the form of an optical diffuser. In principle, it is also possible to use an optical fibre without an outcoupling, which provides a highly divergent beam.

FIG. 7d illustrates a waveguide arrangement 30 having a waveguide tip assembly 34 which directionally redirects optical radiation 33 from the axial direction of the waveguide arrangement 30 to a transverse delivery orientation in a radial direction.

FIG. 7e and FIG. 7f provides different cross-sectional views of another waveguide arrangement 30 for delivering optical radiation.

FIG. 7e provides a view in a cross-sectional plane perpendicular to the axial direction of the waveguide arrangement 30, whereas FIG. 7f provides a view in a cross-sectional plane overlapping with the axial direction of the waveguide arrangement 30 and the tubular element 13.

The illustrated waveguide arrangement 30 comprises an optical fibre 35 situated in an optical fibre ferrule 36 which in turn is surrounded by a waveguide arrangement casing 37. At the second end 32 of the waveguide arrangement 30, the waveguide arrangement 30 is attached to a cone-shaped mirror 34. Optical radiation 33 transmitted through the optical fibre 35 illuminates this cone-shaped mirror 34 such that the optical radiation 33 is circumferentially redirected radially.

FIG. 7g and FIG. 7h provides different cross-sectional views of an additional waveguide arrangement 30 for delivering optical radiation.

FIG. 7g provides a view in a cross-sectional plane perpendicular to the axial direction of the waveguide arrangement 30, whereas FIG. 7h provides a view in a cross-sectional plane overlapping with the axial direction of the waveguide arrangement 30 and the tubular element 13.

The illustrated waveguide arrangement 30 comprises an array of optical fibres 35 aligned in the axial direction of the waveguide arrangement and distributed in a circular arrangement within the waveguide arrangement 30. The centre of the waveguide arrangement 30 has an inner waveguide rod 38 around which the array of optical fibres 35 are arranged. Optical radiation 33 transmitted through the optical fibres 35 illuminates this cone-shaped mirror 34 such that the optical radiation 33 is circumferentially redirected radially. Optionally, optical radiation is only transmitted through a subset of the optical fibres 35.

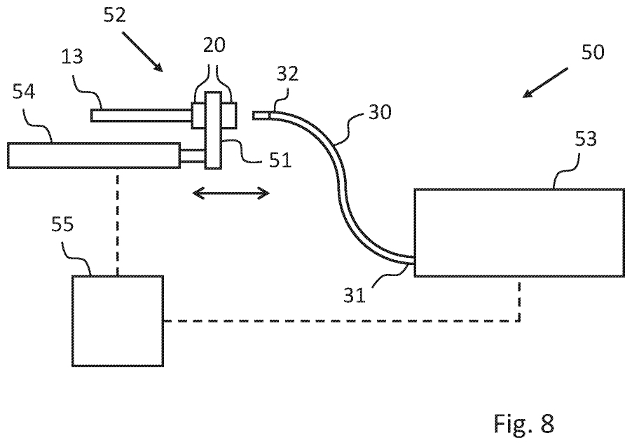

FIG. 8 schematically illustrates a manufacturing apparatus 50 for attaching a connector 13 element to a tubular element 20 in a urinary catheter.

The manufacturing apparatus 50 comprises a catheter fixture 51 configured to hold a catheter preassembly 52. In this example, the catheter preassembly 52 comprises the connector element 20 preliminarily attached to the tubular element 13 by a press fit, and the catheter fixture clamps onto the connector element 20.

The manufacturing apparatus 50 further comprises an optical radiation source 53 and an elongated waveguide arrangement 30 extending between a first end 31 and a second end 32. The first end 31 is optically coupled to the optical radiation source 53. Further, the optical radiation source 53 is configured to emit optical radiation, such that this optical radiation is transmitted through the waveguide arrangement 30 and delivered at the second end 32.

The catheter fixture 51 can be linearly actuated by a relative actuator 54. Accordingly, the catheter preassembly 52 can be moved such that the second end 32 of the waveguide arrangement 30 is inserted into the interior lumen of the tubular element 13. In the illustration, this motion is indicated by a horizontal arrow below the fixture 51. The relative actuator 54 and the optical radiation source 53 is controlled by a controlling arrangement 55, which in this example is a programmable logic circuit. The controlling arrangement 55 is configured to control the relative actuator 54 to insert the second end 32 of the waveguide arrangement 30 into the interior lumen of the tubular element 13 when the catheter preassembly 52 is held in the catheter fixture 51 and configured to subsequently control the optical radiation source 53 to transmit the optical radiation through the waveguide arrangement 30 to deliver the optical radiation to the inner passage surface of the connector element 20. Accordingly, the second polymeric material at the inner passage surface of the connector element 20 is at least partly melted to thereby join at least a part of the inner passage surface to at least a part of the outer surface of the tubular element 13.

Examples, and features of the various exemplary examples described in this application, may be combined with each other (“mixed and matched”), unless specifically noted otherwise.

1.-30. (canceled)

31. A method for attaching a connector to a tube of a urinary catheter, the method comprising:
providing a catheter tube extending in an axial direction between a proximal portion and a distal portion and comprising a first polymeric material, the catheter tube comprising an inner surface and an outer surface, where the inner surface defines an interior lumen of the catheter tube that extends in the axial direction to an opening formed in the distal portion of the catheter tube;
providing a drainage connector comprising a second polymeric material and forming a conduit between a connector opening and a receiving passage of the drainage connector, where the receiving passage forms an inner passage surface;
providing a waveguide configured to guide optical radiation along a length of the waveguide between a first end to a second end of the waveguide and to emit the optical radiation from the second end of the waveguide;
inserting the distal portion of the catheter tube into the receiving passage of the drainage connector and forming an interface between the outer surface of the catheter tube and the inner passage surface of the drainage connector;
transmitting the optical radiation through the waveguide and out of the second end of the waveguide;
delivering the optical radiation to the interface between the outer surface of the catheter tube and the inner passage surface of the drainage connector, melting the second polymeric material of the drainage connector at a location of the inner passage surface, and joining the drainage connector to the catheter tube.

32. The method of claim 31, further comprising inserting the waveguide into the connector opening to an axial location of the interface between the outer surface of the catheter tube and the inner passage surface of the drainage connector and emitting the optical radiation from the second end of the waveguide to the interface between the outer surface of the catheter tube and the inner passage surface of the drainage connector.

33. The method of claim 31, further comprising joining the drainage connector to the catheter tube by continuously welding the drainage connector circumferentially around the outer surface of the catheter tube.

34. The method of claim 31, further comprising:
inserting the waveguide into the connector opening;
rotating the waveguide, emitting the optical radiation from the second end of the waveguide, and continuously welding the drainage connector circumferentially around the outer surface of the catheter tube.

35. The method of claim 31, further comprising:
emitting the optical radiation from a diode laser at a wavelength in a range from 808 nm to 940 nm from the second end of the waveguide.

36. The method of claim 31, further comprising:
inserting the waveguide into the connector opening; and
emitting the optical radiation from a Nd:YAG laser from the second end of the waveguide.

37. The method of claim 31, further comprising:
inserting the waveguide into the connector opening; and
emitting the optical radiation from a CO2 laser from the second end of the waveguide.

38. The method of claim 31, wherein the distal portion of the catheter tube has a diameter larger than a diameter of the receiving passage of the drainage connector, the method further comprising inserting the distal portion of the catheter tube into the receiving passage of the drainage connector and friction-fitting the distal portion of the catheter tube to the drainage connector.

39. The method of claim 31, wherein the first polymeric material and the second polymeric material are both thermoplastic polymers, the method further comprising melting the thermoplastic polymer of the second polymeric material of the drainage connector to the outer surface of the catheter tube.

40. The method of claim 31, wherein the second end of the waveguide is conically shaped, the method further comprising emitting the optical radiation from the conically shaped second end of the waveguide and circumferentially redirecting the optical radiation radially around the interface between the outer surface of the catheter tube and the inner passage surface of the drainage connector.

41. The method of claim 31, wherein the second polymeric material of the drainage connector comprises an optically absorbent additive to provide the inner passage surface of the receiving passage with an optical absorption band, the method further comprising transmitting the optical radiation out of the second end of the waveguide at wavelength selected within the optical absorption band.

42. The method of claim 31, wherein the second polymeric material of the drainage connector comprises an optically absorbent additive to provide the inner passage surface of the receiving passage with an optical absorption band in a range from 800 nm to 1200 nm, the method further comprising transmitting the optical radiation out of the second end of the waveguide at wavelength of 940 nm.

43. The method of claim 31, wherein the second polymeric material of the drainage connector comprises an optically absorbent additive to provide the inner passage surface of the receiving passage with an optical absorption band in a range from 800 nm to 1200 nm, the method further comprising transmitting the optical radiation out of the second end of the waveguide at a spatial peak intensity in a range from 20-200 W/cm2.

44. The method of claim 31, further comprising placing the waveguide outside of the catheter tube and directing the optical radiation from the second end of the waveguide through the catheter tube and to the interface between the outer surface of the catheter tube and the inner passage surface of the drainage connector.

45. The method of claim 31, further comprising:
providing an optically transparent catheter tube and providing an optically opaque drainage connector;
transmitting the optical radiation out of the second end of the waveguide and through the optically transparent catheter tube; and
absorbing the optical radiation with the optically opaque drainage connector and melting the second polymeric material of the drainage connector at the interface between the outer surface of the catheter tube and the inner passage surface of the drainage connector.
